(19) United States
(12) Patent Application Publication
(10) Pub. No.: US 2019/0201417 A1
(43) Pub. Date: Jul. 4, 2019

(54) TOPICAL STEROID THERAPY FOR MUCOSAL AND DERMATOLOGICAL INFLAMMATORY DISEASE

(71) Applicant: CINRX PHARMA, LLC, Cincinnati, OH (US)

(72) Inventors: Drore EISEN, Cincinnati, OH (US); Piyush PATEL, Garnet Valley, PA (US); Catherine PEARCE, Cincinnati, OH (US); Jon ISAACSOHN, Cincinnati, OH (US)

(21) Appl. No.: 16/316,856

(22) PCT Filed: Jan. 3, 2017
(86) PCT No.: PCT/US17/12010

§ 371 Date: Jan. 10, 2019

Related U.S. Application Data
(60) Provisional application No. 62/274,464, filed on Jan. 4, 2016.

(51) Int. Cl. A61K 31/573; A61K 47/10; A61K 47/32; A61K 9/00; A61K 9/06; A61P 1/04; A61P 17/00

(52) U.S. Cl. CPC A61K 31/573; A61K 47/10; A61K 47/32; A61P 17/00; A61K 9/06; A61P 1/04; A61K 9/0014; A61K 9/006

(57) ABSTRACT

A formulation of a topical steroid for therapy of a mucosa or dermatological lesion, such as but not limited to a lichen planus lesion or an aphthous ulcer.

This application claims priority to U.S. Ser. No. 62/274,464 filed Jan. 4, 2016, which is expressly incorporated by reference herein in its entirety.

Lichen planus (LP), including oral lichen planus (OLP), is a chronic inflammatory disease with possible genetic and immunologic causes afflicting the skin and mucosa, and lesions may be visible on skin, scalp, nails, genitals, and mucus membranes. Diagnosis is typically by biopsy of the afflicted area. The oral disease is clinically characterized by bilateral white striations, papules, or plaques on the buccal mucosa, tongue, and gingivae. Painful erythematous, erosive, or bullous lesions and blisters are frequently present.

Current therapy is primarily symptomatic and palliative only, typically involving proper hygiene and eliminating as much as possible symptoms and signs of the disease with medications.

One therapy involves eliminating or reducing local exacerbating factors or changing a drug regimen. For example, broken restorations or prostheses that may cause physical trauma to areas of erythema or erosion should be repaired. Calculous deposits should be reduced by scaling and sharp edges should be filed. For a patient with an isolated plaque-like or erosive lesion on the buccal or labial mucosa adjacent to a dental restoration, allergy testing may determine if a lesion may heal by removing or replacing an allergenic restorative material. For patients on systemic drug therapy, e.g., non-steroidal anti-inflammatory drugs, changing to another drug may help alleviate some or eliminate all symptoms.

All patients with OLP have an increased risk of oral squamous cell carcinoma, the most common of all oral malignancies.

Different patterns of OLP may be seen. A reticular pattern is commonly found on the cheeks as slightly raised lacy, web-like, white threads, termed Wickham's Striae. A plaque-like form appears as a dense thickening of the mucosa. An erythematous (atrophic) pattern can affect any mucosal surface, including the cheeks, tongue, and gums, appearing bright red due to the loss of the top mucosal layer and resulting in discomfort while eating and drinking. Ulcerations can result in pain even when not eating or drinking.

Currently, there is no curative therapy. However, ulcerative and atrophic lesions can be converted into asymptomatic reticular or plaque lesions and often completely eliminated with topical corticosteroid treatment.

Corticosteroids are the most commonly used drugs currently for LP, although they are not approved by a drug regulatory authority for treatment in the mouth. It is common medical practice, however, to prescribe a topical agent and instruct a patient to manually deliver a dose to affected areas in the mouth, often many times per day. Such an option is intended to modulate inflammation and immune response by reducing the lymphocytic exudate and stabilizing the lysosomal membrane. Topical midpotency corticosteroids such as triamcinolone acetonide, high-potent fluorinated corticosteroids such as fluocinonide acetonide, disodium betamethasone phosphate, and super potent halogenated corticosteroids such as clobetasol are used based on the severity of the lesion. Clobetasol belongs to U.S. Class I, Europe Class IV of the corticosteroids, making it one of the most potent steroids available. Steroids are considered first-line treatment, even though they can cause secondary candidiasis, nausea, dry mouth, sore throat, and swollen mouth, and often taste bad.

Other substances or devices by topical administration are used when the first line approach is refractory. These can include calcineurin inhibitors (i.e., cyclosporine, tacrolimus, and pimecrolimus), retinol with its synthetic and natural analogues (retinoids), or aloe vera. In patients with refractory disease, systemic immunosuppressants and anti-inflammatory agents including steroids, methotrexate, mycophenolate, mofetil, azathioprine, hydroxychloroquinine sulfate, and TNF-alpha inhibitors (adalimumab, etanercept) may be useful.

Along with lack of controlled trials for safety and efficacy with these agents in the mouth, a disadvantage with current preparations is the difficultly of adherence to the mucosa, and subsequent non-predictability and non-uniformity of the steroid's effect. Patients do not have a user friendly device that can deliver an adherent or “sticky” form of the drug to the oral mucosa, where topical corticosteroids are often washed away by saliva or inadvertently swallowed before optimal therapeutic effects can be achieved.

Steroid delivery to the mouth has included a steroid preparation in an adhesive paste to treat gingival lesions, and applying corticosteroid ointment topically to mucosal lesions using cloth strips. Other methods are known, e.g., Aleinikov et al. (1996), Topical steroid therapy in oral lichen planus: review of a novel delivery method in 24 patients. J Can Dent Assoc 62(4): 324-327.

### BRIEF DESCRIPTION OF THE DRAWINGS

FIG. 1 is a schematic view of an embodiment of a spray bottle applicator having a nozzle for directing the pharmaceutical composition to a treatment site.

FIG. 2 is a schematic view of an embodiment of an applicator configured to deliver the pharmaceutical compositions to the treatment site in the form of a puff of dry powder.

FIG. 3 is a schematic view of an applicator that includes a gel tube coupled to an applicator tip for dispensing the pharmaceutical compositions.

FIG. 4 is a schematic view of a tube with a child-resistant cap. FIG. 5 is a schematic view of an embodiment of an oral dispensing syringe for dispensing the pharmaceutical compositions.

FIG. 6 is a schematic view of an embodiment of a gel can and paddle applicator for applying the pharmaceutical compositions to a treatment site.

FIG. 7 is a schematic view of an embodiment of a thin film supporting or containing the pharmaceutical compositions for application to a treatment site.

FIG. 8 is a schematic view of an embodiment of a biodegradable mucoadhesive drug-loaded polymer mesh for applying the pharmaceutical compositions to a treatment site.

FIG. 9 is a schematic view of an embodiment of a mucoadhesive tablet for applying the pharmaceutical compositions to a treatment site.

FIG. 10 is a schematic view of an embodiment of a unit dose pouch, flexible bottle or plastic container with a molded nozzle and a break-off tab for dispensing a cream, lotion, gel, or ointment to a treatment site.

One embodiment of the invention is a method for delivering a topical steroid, the method providing long-acting, sustainable properties, taste-masking properties, and suitable for use in either acute or chronic administration for therapy of LP. The topical steroid may be delivered by the inventive method in the mouth, to the skin, or to a mucosal surface.

One embodiment of the invention is a composition of bioadhesive polymers that provide steroid in a long-acting preparation, the composition adhering to the cheeks, tongue, gums, and other areas in the mouth where OLP lesions are present or may be present. In a specific embodiment, the composition is formulated as a lozenge, tablet, or powder. In another specific embodiment, the composition is formulated as a cream, gel, or ointment that could be delivered by a bottle, syringe, tube, or a unit dose container. In another specific embodiment, the composition is formulated as a gum. In another specific embodiment, the composition is formulated as a mouthwash.

One embodiment of the invention is a spray device for steroid delivery.

The invention provides patient-friendly, optimally delivered formulations of a highly potent corticosteroid for acute and chronic treatment of any mucosal or dermatological pathology. In one embodiment, the pathology being treated is LP. In one embodiment, the pathology being treated is an aphthous ulcer. In one embodiment, the pathology being treated is one or more of recurrent aphthous ulcers such as aphthous stomatitis (canker sores); a vesiculoerosive disease such as pemphigus, pemphigoid, and graft vs. host disease; glossitis; stomatitis of any etiology including allergic contact or irritant contact; systemic hypersensitivity to medications, localized inflammatory conditions including geographic tongue and lichenoid mucositis, etc. In one embodiment, the pathology is pruritis, proctitis, vaginitis, etc. In one embodiment for oral administration, the formulation is a flavored and taste-masked mucosal spray that adheres to the tongue, gums, and inner mucosal surface of the cheek where OLP commonly appears. The preparation can be administered once a day to three times a day.

The disclosed spray composition adheres effectively to lesions such as those caused by an aphthous ulcer, OLP, etc. and thus minimizes commonly associated drawbacks of current therapies, such as bad taste and suboptimal adherence of the medication to cheek, tongue, lips, and gums. One embodiment is a method of use of a uniquely delivered pharmaceutical composition, which includes a taste-masked topical steroid, in the mouth that has long-acting, sustainable properties, and can be used for acute or chronic administration in OLP. The disclosed pharmaceutical compositions include bioadhesive polymers to provide the pharmaceutically active agent, specifically a steroid, in a long-acting preparation with the ability to adhere to cheeks, tongue, gums and other areas in the mouth containing OLP lesions. In embodiments, the pharmaceutical compositions may be incorporated into a mucosal spray or may be incorporated into lozenges, creams, bioadhesive creams, gels, ointments, powders, long-acting gums, and/or long-acting mouthwash. By providing a pleasant tasting spray that adheres effectively to the lesions, the disclosed pharmaceutical compositions minimizes the issues commonly associated with current therapies, such as bad taste and poor adherence of medication to the cheek, tongue, and/or gums.

In one embodiment, the composition is a formulation of two concentrations of clobetasol: 0.5% w/w clobetasol and 0.025% w/w clobetasol. In other embodiments, any combination of mid-high potency corticosteroids alone or in combination with anesthetic agents, such as lidocaine or another topical numbing/pain agent, can be used. Other preparations may include calcineurin inhibitors, antioxidants, and other therapeutically or pharmaceutically active agents.

Methods and pharmaceutical compositions for alleviating the symptoms of and inflammation associated with LP formulated for topical administration to mucosal surfaces. Currently there are a small number of drugs that are routinely delivered via the oral mucosa, such as systemic delivery of glyceryl trinitrate for angina relief and topical corticosteroid administration for OLP. Hearnden et al., New developments and opportunities in oral mucosal drug delivery for local and systemic disease. Advanced Drug Delivery Reviews, 64(2012), 16-28. doi:10.1016/j.addr.2011.02.008.

If not specified, percentages refer to weight/weight.

The disclosed pharmaceutical compositions, delivery methods, and treatment methods provide patient-friendly, optimally delivered formulations of a highly potent corticosteroid for the acute and chronic treatment of oral lichen planus. In one embodiment, the formulation is a flavored and/or taste-masked mucosal spray that adheres to inside cheeks, tongue, lips, and gums where lesions commonly appear. The compositions can be administered once to three times per day.

The disclosed compositions and methods use mucoadhesive delivery technologies for safe and efficacious delivery of a corticosteroid, such as clobetasol, in the mouth. These mucoadhesive delivery technologies include all methods of diffusion in the oral mucosa: (i) passive diffusion including trans-cellular (through cells) and para-cellular (where material passes through lipid rich domains around the cells), (ii) carrier mediated transport, and (iii) endocytosis/exocytosis where material is actively taken up and excreted by cells via the endocytic pathway.

Mucous membranes (mucosae) line various cavities of the body that are either externally exposed to the environment or are internal organs. The oral mucosa is the mucous membrane lining the inside of the mouth and consists of stratified squamous epithelium (oral epithelium) and an underlying connective tissue (lamina propria). It can be further divided into three main categories based on function and histology: masticatory mucosa consisting of keratinized stratified squamous epithelium found on the dorsum of the tongue, hard palate and attached gingiva; lining mucosa consisting of non-keratinized squamous epithelium found almost everywhere else in the oral cavity including the buccal mucosa which lines the cheeks, the labial mucosa which is the inside lining of the lips, and the alveolar mucosa which is the mucosa between the gums and the buccal/labial mucosa; and specialized mucosa in the regions of the taste buds on lingual papillae on the dorsal surface of the tongue.

As previously stated the symptoms of OLP include bilateral white striations, papules or plaques on the buccal mucosa, tongue, and gingiva thus involving all three categories of the oral mucosa.

The symptoms of aphthous stomatitis include one or more discreet shallow painful ulcer(s) on non-keratinizing epithelial surfaces in the mouth, i.e., anywhere except the attached gingiva, the hard palate, and the dorsum of the tongue; more severe forms may also involve keratinizing epithelial surfaces. Aphthous stomatitis is common and affects about 20% of the population to some degree. Recurrent aphthous ulcer minor is the most common form, and typically heals without scarring. Periodicity varies among individuals. Recurrent aphthous ulcer major accounts for 5-10% of cases and presents as a single deep ulcer, although multiple ulcers can occur. Healing may take weeks to months; scarring usually results. The rare herpetiform recurrent aphthous ulcer occurs in clusters that may be small and localized, or distributed throughout the soft mucosa of the oral cavity. These occur predominantly on un-keratinized mucosa.

Bioadhesive polymers adhere to any moist surface, thus the mucoadhesive/bioadhesive formulation will adhere to both saliva-moistened keratinized and non-keratinized mucosa.

Using buccoadhesive drug delivery, the drug is easily administered and administration can be readily halted if required; there is drug release for a long period; drug can be administered to patients who cannot self-administer; the drugs bypass first pass metabolism thus increasing bioavailability; drugs that are otherwise unstable in the acid stomach environment can be administered; drug is absorbed by passive diffusion; the physical state, shape, size, and surface can be customized or changed; the absorption rate is maximized because of the close contact with the absorbing mucosa; and onset of action is rapid.

The mucosa, a vascular tissue that is less prone to irritation, is also a hydrated environment that assists in steroid solubilization. A solubilized steroid desirably has increased bioavailability, maintaining a required level of the pharmaceutical agent in the oral cavity for extended time intervals and improving drug availability.

Vaginal mucosa is similar to oral mucosa; the vaginal wall from the lumen outwards has a mucosa of non-keratinized stratified squamous epithelium with an underlying lamina propria of connective tissue. Anal mucosa differs in its rectal columnar mucosa becoming stratified squamous and non-keratinized within the anal canal (anoderm) from the dentate line, then keratinized at the anal verge. The upper anal canal (has longitudinal folds or elevations of tunica mucosa, lined by simple columnar epithelia. Its lower ends are joined together by folds of mucous membrane termed anal valves.

In use, delivery of the active may be affected by permeability characteristics through various skin or mucosal layers, and by any compromised skin or mucosa state, including by the infective agent. As such, some delivery vehicles or mechanisms may be effective for both mucosal and skin delivery.

In one embodiment, the steroid is delivered in a vehicle or formulation that is lipid soluble, non-ionized, and provides a sustained release profile. For example, a spray device may be used to administer the composition, with an adjustable delivery nozzle so the user can adjust or position the agent to directly contact the lesion in use. The compositions may also be administered as a solid powder using a powder canister. The disclosed compositions may be incorporated into other delivery mechanisms or formulations, e.g., liquids, gels, mucoadhesive tablets, long lasting gums, mouthwashes, lozenges, bioadhesive creams, mucoadhesive drug-loaded polymer meshes, drug-polymers blends, polymer-coated microparticles and/or nanoparticles, drug encapsulated liposomes, etc., and may be administered using an oral dispensing syringe, a paddle-type applicator, a gel-tube applicator, etc.

The pharmaceutical compositions disclosed herein generally include a therapeutic amount of one or more pharmaceutically active agents and at least one excipient added to improve the coating of the pharmaceutical composition to a treatment site. In one aspect, the excipient can include a mucoadhesive or bioadhesive, which can increase the duration of contact between the pharmaceutically active agent and the oral mucosa and the absorption of the active agent by the absorption surface. The absorption surface is the tissue surface at a treatment site underneath the oral mucosa to which the pharmaceutically active agent is intended to be applied. The pharmaceutical compositions can be applied in the form of ointments, creams, lotions, gels, powders or pastes, and can be applied to treatment sites with or without occlusion by films or tapes or via specific adhesive bandages. The compositions can also include a vehicle to facilitate administration of the composition to the OLP site and one or more other excipients, examples of which may include, but are not limited to, binders, fillers, solvents, lubricants, antioxidants, buffering agents, salts, surfactants, vitamins, pigments, flavorants, disintegrated agents, plasticizers, or combinations thereof.

In one embodiment, the steroid composition may include a topical anesthetic, e.g., benzocaine, lidocaine, capsaicin, or any others known in the art. In one embodiment, the steroid composition includes lidocaine, capsaicin, and resiniferotoxin.

A human or other mammal afflicted with LP, OLP, or other conditions suitably treatable with a topical corticosteroid, can be treated through periodic topical application of the pharmaceutical compositions disclosed herein one or more times daily. For OLP therapy, the pharmaceutical compositions can be applied directly to the oral mucosa at treatment sites positioned in the buccal or sublingual regions of the mouth. The pharmaceutical compositions can also be incorporated into mouth washes, lozenges, or gums, which can be periodically used to dispense the pharmaceutical compositions to the treatment sites for limited or extended periods of time.

The pharmaceutical agent may be a corticosteroid used alone or in combination with one or more other pharmaceutical agents, such as an anesthetic agent. The corticosteroid may be a topically active corticosteroid. Examples of corticosteroids may include, but are not limited to, one or more of aclometasone, amcinomide, beclometasone, betamethasone, budesonide, ciclesonide, clobetasol, clobetasone, clocortolone, cloprednol, cortivazol, deflazacort, deoxycorticosterone, desonide desoximetasone, dexamethasone, diflorasone, diflucortolone, difluprednate, fluclorolone, fludrocortisone, fludroxycortide, flumetasone, flunisolide, fluocinolone acetonide, fluocinonide, fluocortin, fluocortolone, fluorometholone, fluperolone, fluticasone, fluticasone propionate, fuprednidene, formocortal, halcinonide, halometasone, hydrocortisone aceponate, hydrocortisone buteprate, hydrocortisone butyrate, loteprednol, medrysone, meprednisone, methylprednisolone, methylprednisolone aceponate, mometasone furoate, paramethasone, prednicarbate, prednisone, prednisolone, prednylidene, remexolone, tixocortol, triamcinolone and ulobetasol, and combinations, pharmaceutically acceptable salts and esters thereof. In one embodiment, the pharmaceutical composition includes a therapeutic amount of clobetasol.

In one embodiment, a corticosteroid may be used in conjunction with a topical anesthetic, such as lidocaine or another topical pain numbing and/or relieving agent. Examples of anesthetic agents may include, but are not limited to, one or more of lidocaine, benzocaine, bupivacaine, articaine, cocaine, etidocaine, flecamide, mepivacaine, pramoxine, prilocalne, procaine, chloroprocaine, oxyprocaine, proparacaine, ropivacaine, tetracaine, dyclonine, dibucaine, chloroxylenol, cinchocaine, dexivacaine, diamocaine, hexylcaine, levobupivacaine, propoxycaine, pyrrocaine, risocaine, rodocaine, and pharmaceutically acceptable derivatives and bioisosteres thereof, as well as combinations thereof.

In one embodiment, the pharmaceutical compositions may include other therapeutically agents, e.g., one or more antifungal agents, antibacterial agents, vitamins, etc.

In one embodiment, one or more of the pharmaceutical agents, including the corticosteroid, incorporated into the pharmaceutical compositions may be incorporated into the composition as particles, such as corticosteroid particles suspended or dispersed in an aqueous medium. In one embodiment, the particles may be microparticles with a mean diameter ranging from about 0.1 microns to about 50 microns, or from about 1 micron to about 30 microns, or from about 2 microns to about 10 microns.

The pharmaceutical agents, including the corticosteroid and any supplemental therapeutic agent, is desired to be present in the composition in an amount constituting a therapeutically effective dose. A therapeutically effective dose is an amount of the pharmaceutical agent that, upon treatment, results in a degree of reduction of symptoms and/or inflammation relative to the status of such symptoms and/or inflammation prior to treatment. The dosage forms and methods of applying dosage forms containing effective amounts are within the scope of the disclosure.

The pharmaceutical compositions can be administered one to three times per day. In one aspect, the pharmaceutical composition can include clobetasol in an amount in a range of about 0.025% by weight to about 0.5% by weight inclusive of the pharmaceutical composition. In one aspect, the pharmaceutical composition includes clobetasol in an amount of about 0.025% of the weight of the pharmaceutical composition. In another aspect, the pharmaceutical composition includes clobetasol in an amount of about 0.5% of the weight of the pharmaceutical composition. A typical dose would be less than one fingertip unit (FTP), which is the amount of topical steroid that is squeezed out from a standard tube along an adult's fingertip and which contains about a gram of steroid. Due to the oral cavity's small size, a typical dose that would cover multiple oral surfaces would be a pea sized amount, equal to between about one-fifth to one-third of a gram of steroid.

Each application would contain about 1 gram, thus for a typical 70 kg adult male the approximate ranges are as follows: clobetasol marketed product is 0.05% w/w=0.5 mg/g=0.5 mg/70 kg=about 7 μg/kg; 0.7 μg/kg×2 (twice a day)=1.4 μg per day. A patient range is about 0.025% w/w to 0.5% w/w; 0.025%=0.25 mg/g=3.5 μg/kg x 2=7 μg per day and 0.5%=5 mg/g=70 μg/kg×2 (twice a day)=140 μg. Clobetasol is a potent corticosteroid so the dosage will be in the pg range. Other less potent corticosteroids would likely be administered at a higher dose.

In one embodiment, a combination of steroids may be used in different formulations, e.g., mid-potency, high potency, and/or super potency steroids with none weaker than mid-potency steroids as defined and known in the art. Exemplary steroids include, but are not limited to, mid-potency steroids triamcinolone acetonide 0.25%-0.5%, betamethasone valerate 0.1%; high potency steroids fluocinonide 0.05%-0.1%, halcinonide 0.1%, desoximetasone 0.05%-0.25%; and super potency halobetasol propionate 0.05%, betamethasone dipropionate 0.05%. While the concentrations provided are those that are commercially available, the concentration range may be expanded.

Exemplary batches that are mixtures of various components, including excipients and actives, are now provided:

Anhydrous formulation examples are as follows:

Batch 1 is (poly)ethylene glycol (PEG) 400, hydroxypropyl methyl cellulose (HPMC)

Batch 2 is oleyl alcohol, HPMC

Batch 3 is glycerin, gelatin, PEG 300

Batch 4 is glycerin, CARBOPOL® 980

Batch 5 is glycerin, CARBOPOL® 974P

Batch 6 is HPMC, glycerin

Batch 7 is glycerin, AVICEL® RC-591

Batch 8 is mineral oil, AVICEL® RC-591

Batch 9 is CARBOPOL® 974P, PEG 300

Batch 10 is AVICEL® RC-591, PEG 300

Batch 11 is HPMC, PEG 300

Batch 12 is pregel starch, PEG 300

Batch 13 is mineral oil, (hydrophobically-modified methyl cellulose (HMMC)

Batch 14 is glycerin, HEC 250 M, PEG 300

Batch 15 is glycerin, pregel starch

Batch 16 is CARBOPOL® 980, PEG 300

Batch 17 is HEC 250 M, PEG 300

Batch 18 is medium chain triglycerides (MCT), PEG 300

Batch 19 is oleyl alcohol, PEG 300

Batch 20 is polysorbate 80, PEG 300

Batch 21 is glycerin, ethlycellulose

Batch 22 is ethlycellulose, PEG 300

Batch 23 is propyl gallate, PEG 300

Batch 24 is BHT, PEG 300

Batch 25 is tertiary butylhydroquinone (TBHQ), PEG 300

Batch 26 is glycerin and poloxamer 407, PEG 300

Batch 27 is glycerin, CARBOPOL® 980, PEG 300, clobetasol propionate

Batch 28 is glycerin, CARBOPOL® 974P, PEG 300, clobetasol propionate

Batch 29 is glycerin, CARBOPOL® 980, PEG 300, clobetasol propionate (0.025%)

Batch 30 is glycerin, CARBOPOL® 974P, PEG 300, clobetasol propionate (0.025%)

Batch 31 is glycerin, CARBOPOL® 980, PEG 300

Batch 32 is glycerin, CARBOPOL® 974P, PEG 300

Batch 33 is glycerin, CARBOPOL® 980, PEG 300, BHA

Batch 34 is glycerin, CARBOPOL® 974P, PEG 300, BHA

Batch 35 is glycerin, CARBOPOL® 980, PEG 300, BHA, clobetasol propionate

Batch 36 is glycerin, CARBOPOL® 974P, PEG 300, BHA, clobetasol propionate

Batch 37 is glycerin, CARBOPOL® 980, PEG 300, clobetasol propionate

Batch 38 is glycerin, CARBOPOL® 974P, PEG 300, clobetasol propionate

Batch 39 is glycerin (different manufacturers), CARBOPOL® 980, PEG 300 (different manufacturers)

Batch 40 is glycerin (different manufacturers), CARBOPOL® 974P, PEG 300 (different manufacturers)

Aqueous formulation examples are as follows:

Batch 1 is water, carrageenan Iota, propylene glycol, PEG 400

Batch 2 is water, carrageenan Kappa, propylene glycol, PEG 400

Batch 3 is water, carrageenan Kappa & Iota, propylene glycol, PEG 400

Batch 4 is water, carrageenan Iota, propylene glycol, PEG 400, HEC 250 HX

Batch 5 is water, carrageenan Kappa, propylene glycol, PEG 400, HEC 250 G

Batch 6 is water, propylene glycol, PEG 400, HEC 250 HX

Batch 7 is water, propylene glycol, PEG 400, HEC 250 G

Batch 8 is water, propylene glycol, PEG 400, HEC 250 M

Batch 9 is water, PEG 400, CARBOPOL® 974P

Batch 10 is water, PEG 400, CARBOPOL® 974P, trolamine

Batch 11 is water, propylene glycol, PEG 400, CARBOPOL® 974P, Trolamine

Batch 12 is water, sodium benzoate, propyl gallate

Batch 13 is water, propylene glycol, PEG 400, sodium alginate

Batch 14 is water carrageenan Kappa & Iota, propylene glycol, PEG 400, sodium benzoate, potassium sorbate

Batch 15 is water, carrageenan Kappa, propylene glycol, PEG 400, HEC 250 G, sodium benzoate, potassium sorbate

Batch 16 is water, propylene glycol, PEG 400, HEC 250 M, CARBOPOL® 974P, trolamine Batch 17 is water, propylene glycol, PEG 400, HEC 250 G, sodium benzoate, propyl gallate, Potassium Sorbate

Batch 18 is water, propylene glycol, PEG 400, HEC 250 G, propyl gallate, potassium sorbate, clobetasol propionate

Batch 19 is water, propylene glycol, PEG 400, HEC 250 M, propyl gallate, potassium sorbate, clobetasol propionate.

Batch 20 is water, PEG 300, CARBOPOL® 974P, BHA, clobetasol propionate.

Exemplary specific formulations comprising clobetasol that may be used are as follows:

Formulation 1:

| | |
|---|---|
| CARBOPOL ® 974P | 1.25% |
| glycerin | 48% |
| BHA | 0.01% |
| clobetasol propionate | 0.05% & 0.025% |
| PEG 300 | qs to 100% |

Formulation 2:

| | |
|---|---|
| CARBOPOL ® 974P | 1.25% |
| glycerin | 48% |
| BHA | 0.01% |
| clobetasol propionate | 0.05% & 0.025% |
| PEG 300 | qs to 100% |
| peppermint oil (flavor) | |

sodium benzoate and potassium sorbate (taste masking agents)

Formulation 3:

| | |
|---|---|
| CARBOPOL ® 974P | 1.25% |
| glycerin | 48% |
| BHA | 0.01% |
| clobetasol propionate | 0.05% & 0.025% |
| PEG 300 | qs to 100% |
| cherry (flavor) | |

sodium benzoate and potassium sorbate (taste masking agents)

Formulation 4:

| | |
|---|---|
| CARBOPOL ® 980 | 1.25% |
| glycerin | 48% |
| BHA | 0.01% |
| clobetasol propionate | 0.05% & 0.025% |
| PEG 300 | qs to 100% |
| peppermint oil (flavor) | |

sodium benzoate and potassium sorbate (taste masking agents)

Exemplary mucoadhesive or bioadhesive excipients are selected from the group consisting of natural, synthetic or biological, polymers, lipids, phospholipids, and the like. Examples of natural and/or synthetic polymers include cellulosic derivatives (such as methylcellulose, carboxymethyl cellulose, hydroxyethyl cellulose, hydroxyethylmethy] microcrystalline cellulose, etc.), natural gums (such as guar gum, xanthan gum, locust bean gum, karaya gum, vee-gum etc), polyacrylates (such as CARBOPOL® polymers, polycarbophil, polymers emulsifiers, etc), alginates, gelatin, thiol-containing polymers, polyoxyethylenes, polyethylene glycols (PEG) of all molecular weights (preferably between 1000 and 40,000 Da, of any chemistry, linear or branched), dextrans of all molecular weights (preferably between 1000 and 40,000 Da of any source), block copolymers, such as those prepared by combinations of lactic & glycolic acid (PLA, PGA, PLGA of various viscosities, molecular weights and lactic-to-glycolic acid ratios) polyethylene glycol-polypropylene glycol block copolymers of any number and combination of repeating units (such as PLURONIC® brand block copolymers, TETRONIC® block copolymers, or GENAPOL® block copolymers), combination of the above copolymers either physically or chemically linked units (for example PEG-PLA or PEG-PLGA copolymers) mixtures. Preferably the bioadhesive material is selected from the group of polyethylene glycols, polyoxyethylenes, polyacrylic acid polymers, such as CARBOPOL® polymers (such as CARBOPOL® 71G, 934P, 971P 974P) and polycarbophils (such as NOVEON® AA-1, CA-1, and CA-2 polycarbophils), cellulose and its derivatives and most preferably it is polyethylene glycol, CARBOPOL® polymers, and/or a cellulosic derivative or a combination thereof. Many of these polymers can be chemically cross-linked with polyalkenyl alcohols or divinyl glycol. Several commercial formulations are designed for topical, oral suspension/solution, bioadhesive and oral care applications. Other applications include gels, lotions, creams and emulsions that can be poured, pumped, spread, or sprayed. In one embodiment, the mucoadhesive agent is a CARBOPOL® polymer, and/or a cellulosic derivative thereof.

Other examples of mucoadhesive or bioadhesive excipients include, but are not limited to, a soluble polyvinylpyrrolidone polymer (PVP), a carbomer homopolymer, a carbomer copolymer, one or more maltodextrin, alginate, a cross-linked alginate gum gel, a water-swellable but water-insoluble fibrous cross-linked carboxy-functional polymer, a hydrophilic polysaccharide gum, thiomers (e.g., thiolated chitosan, thiolated polycarbophil, thiolated alginate, thiolated cellulose derivatives, thiolated carboxymethyl cellulose, thiolated polyacrylic acid, or thiolated polyacrylates), lectin, hydroxpropyl methyl cellulose (HPMC), cellulose derivatives, N-(2-hydroxypropyl)methacrylamide (HPMA) copolymers, a water-dispersible polycarboxylated vinyl polymer, cationic polymers, non-ionic polymers, or anionic polymers. Cationic polymers include but are not limited to chitosan, including chitosan soluble in dilute aqueous acids, chitosan (Wella “low viscosity”), chitosan (Wella “high viscosity”), chitosan (Dr. Knapczyk), daichitosan H, daichitosan VH, Sea Cure 240, Sea Cure 210+, chitosan (Sigma), polycarbophil/diachitosan VH blend; DEAE-dextran, and aminodextran. Non-ionic polymers include but are not limited to Scleroglucan, He-starch, HPC. Anionic polymers include but are not limited to sodium carboxymethylcellulose (CMC) (low viscosity), sodium CMC (medium viscosity), sodium CMC (high viscosity), pectin, xanthan gum, locust bean gum, and polycarbophil. Other examples of mucoadhesive polymers that can be used include, but are not limited to, poly(acrylic acid-co-acrylamide), polyisoprene (PIP), polyisobutylene (PIB), semi-natural and natural polymers, e.g., agarose, chitosan, gelatin, hyaluronic acid, various gums such as guar gum, xanthan, gellan, carragenan, pectin, and sodium alginate; and synthetic polymers, e.g., cellulose derivatives such as carboxy methyl cellulose (CMC), thiolated CMC, NaCMC, hydroxyethylcellulose (HEC), hydroxypropyl cellulose (HPC), hydroxypropylmethylcellulose (HPMC), and MC; poly(acrylic acid)-based polymers such as carbapol (CP), polycarbophil (PC), polyacrylic acid (PAA), polyacrylates, poly(methyl vinyl ether-co-methacrylic acid), poly(2-hydroxy ethyl methacrylate), poly(acrylic acidco-ethyl hexyl acrylate), poly(methacrylate), poly(isobutylcyanoacrylate), copolymer of acrylic acid and PEG]; and other synthetic polymers such as polyoxyethylene, PVA, poly(vinylpyrrolidione) PVP, and thiolated polymers; water-soluble polymers, e.g., CP, HEC, HPC, HPMC (cold water), PAA, NaCMC, sodium alginate, and water-insoluble polymers, e.g., chitosan (soluble in dilute aqueous acids), ethyl cellulose (EC), and PC; cationic polymers such as aminodextran, chitosan, (DEAE)-dextran, and TMC; anionic polymers, e.g., chitosan-ethylene diamine tetraacetic acid (chitosan-EDTA), CP, CMC, pectin, PAA, PC, sodium alginate, NaCMC, and xanthan gum; non-ionic polymers, e.g., hydroxy ethyl starch, HPC, poly(ethylene oxide), and PVA; covalently bonded polymers, e.g., PVP and scleroglucan; hydrogen bonded polymers such as cyanoacrylate; bioadhesive polymers, e.g., acrylates such as hydroxylated methacrylate, poly(methacrylic acid)], CP, PC, PVA, and chitosan. Still other examples of bioadhesive polymer formulations include chitosan and sodium alginate; chitosan, polycarbophil, sodium alginate, gellan gum; CP, HPMC, PC, sodium carboxymethylcellulose (SCMC), polyacrylic acid (PAA); HPMC and CP 934; HPMC and PC; CP, HPMC, PC, SCMC, and PAA; CP 934 and PVP K-30; CP, HPMC, PC, SCMC, and PAA; carbomer and HPMC; HPC-M and CP 934; HPC and CP 934; HPMC and poly(acrylicacid-2-5-dimethyl 1-5 hexadiene) PADH; CP-934 and HPC-H; CP-934 and HPMC; modified starch and PAA; modified starch and CP-934; CP-934P and HPMC; hakea gum; sodium alginate, HPMC, CP-934P, and PC; HPC, CP-934P, PVP; CP 974P, HPMC and K4M; anionic, cationic, and nonionic polymers; cross linked PAA and HPC; CP 974 HPMCK4M, sodium alginate and HPMC; HPMC (methocelk4m), carbapol934P, polycarbiphyl; CP 934P and CMC; polycarbophil and CP 934P; HEMA and polymeg; carbomer and HPMC; and CP-934P and HPMC K4M.

Chitosan, due to its mucoadhesive character (Lehr et al., 1992) and favorable toxicological properties, is a potential absorption enhancer across intestinal epithelia. Chitosan glutamate can reduce transepithelial electrical resistance (TEER) in vitro of a cultured intestinal epithelial cell line (Caco-2) (Borchard et al. 1996). Chitosan glutamate was able to increase the transport of hydrophilic molecules such as [14C]mannitol [molecular weight (MW) 182.2] and a fluorescein-dextran (MW 4400) significantly in Caco-2 cell monolayers (Artursson et al., 1994; Borchard et al., 1996; Schipper et al., 1996). Similarly, the transport of the peptide drug 9-desglycinamide-8-arginine vasopressin (DGAVP, MW 1412) was increased markedly after coadministration with chitosan glutamate in Caco-2 cell monolayers (Luessen et al., 1997). Chitosan salts such as chitosan glutamate and chitosan hydrochloride have been used in vivo as absorption enhancers for peptide drugs. The nasal application of insulin with chitosan glutamate led to a significant reduction in blood glucose levels of rats and sheep (Ilium et al. 1994), and the intraduodenal application of buserelin (MW 1299.5) and chitosan hydrochloride in a gel formulation increased the absolute bioavailability of buserelin from 0.1±0.1 to 5.1±1.5% (Luessen et al. 1996a). These increases in absorption could be attributed to the effect of chitosan on the integrity of the epithelial tight junctions. Tight junctions play a crucial part in maintaining the selective barrier function of cell membranes and in sealing cells together to form a continuous cell layer through which even small molecules cannot penetrate. However, tight junctions are permeable to water, electrolytes, and other charged or uncharged molecules up to a certain size (Madara, 1989; Wilson and Washington, 1989). Tight junctions respond to changes in calcium concentrations, cyclic AMP (cAMP), osmolarity, pH, and the status of the cytoskeleton (Cereijido et al. 1993).

Chitosan salts may open the tight junctions in a concentration- and pH-dependent way to allow paracellular transport of large hydrophilic compounds. The increase in the transport of these compounds could be attributed to an interaction of a positively charged amino group on the C-2 position of chitosan with negatively charged sites on the cell membranes and tight junctions of the mucosal epithelial cells to allow opening of the tight junctions. Chitosan glutamate has been demonstrated to induce changes in the F-actin distribution (Artursson et al. 1994). It is also known that pharmacological agents that interact with cytoskeletal F-actin simultaneously increase the paracellular permeability (Meza et al. 1982). This is in agreement with the hypothesis that F-actin is directly or indirectly associated with the proteins in the tight junctions such as zonula occludens-1 (ZO-1) (Madara 1987). Chipper et al. (1997) have shown that chitosan induces a redistribution of cytoskeletal F-actin and the tight junction protein ZO-1. Confocal laser scanning microscopy has confirmed that chitosan is able to open the tight junctions to allow the paracellular transport of large hydrophilic compounds (Borchard et al., 1996; Schipper et al. 1997). Mucoadhesion may play an additional role in this process by increasing the residence time of the drugs on the cell surfaces.

In all these studies, absorption enhancement was found only in acidic environments and the charge density of chitosan influences the enhancement of mucosal transport.

Transepithelial electrical resistance (TEER) is a good indication of the tightness of the junctions between cells and can predict the paracellular transport of hydrophilic compounds. For example, Kotze et al (p. 346-349) discloses that chitosan hydrochloride and chitosan glutamate decreases TEER of cultured intestinal epithelial cell lines (Caco-2) in an acidic environment to allow the paracellular transport of a hydrophilic marker. Hence, TEER reduction may be attributed to an interaction of various chitosan polymers (chitosan glutamate, chitosan hydrochloride) with the cell surfaces or the light junctions and may enhance absorption of peptide drugs (Kotje et al. pp. 346-352). Other penetration enhancers include surfactants by perturbing intercellular lipids and protein domain integrity, e.g., anionic surfactants such as sodium lauryl sulfate, cationic surfactants such as cetyl pyridinium choride, and nonionic surfactants such as Poloxamer, Brij, Span, Myrj, and Tween; bile salts by perturbing lipid and protein domain integrity, e.g., sodium glycol deoxycholate, sodium glycocholate, sodium tauro deoxycholate, and sodium tauro cholate; fatty acids by increasing fluidity of phospholipid domains, e.g., oleic acid, caprylic acid, lauric acid, lyso phosphatidyl choline, and phosphatidyl choline; cyclodextrins by inclusion of membrane compounds, e.g., a, β, y cyclodextrin, methylated β-cyclodextrins; chelators by interfering with calcium ions, e.g., EDTA, citric acid, sodium salicylate, and methoxy salicylates; positively charged polymers by ionic interaction with a negative charge on the mucosal surface, e.g., chitosan and trimethyl chitosan; and cationic compounds by ionic interaction with a negative charge on the mucosal surface, e.g., poly-L-arginine and L-lysine.

The excipient for the mucoadhesive/bioadhesive may be various forms, e.g., liquid, solid, gel, lotion, etc. and is typically present in a range of about 1% to about 50% w/w, preferably in a range of about 1% to about 40% w/w or most preferably in a range of about 2 to about 30% w/w. In one embodiment, a formulation may contain one or more different mucoadhesives or bioadhesives in any combination. Bioadhesion increases the residence time of a dosage form at the absorption site, and thus may result in increased drug bioavailability.

The bioadhesive also protects the drug from being washed away from saliva or swallowed before optimal therapeutic effects are achieved. Buccal absorption occurs by passive diffusion of the non-ionized species, governed primarily by a concentration gradient, through the intercellular spaces of the epithelium. The passive transport of non-ionic species across the lipid membrane of the buccal cavity is the primary transport mechanism. The buccal mucosa has been said to be a lipoidal barrier to drug passage, as with many other mucosal membranes, and the more lipophilic the drug molecule, the more readily it is absorbed. Buccal drug absorption dynamics may be described by first order rate processes. Potential barriers to buccal drug absorption include salivary secretion, altering buccal absorption kinetics by changing the dryg concentration in the mouth.

Bioadhesion, also known as mucoadhesion, defines the ability of a biological or synthetic material to “stick” to a mucous membrane, resulting in adhesion of the material to the tissue for a protracted period of time. This ability provides application in drug delivery and enhanced drug bioavailability that results from the lengthened period of time in which the bioadhesive dosage form is in contact with the absorbing tissues, versus a standard dosage form. For a material to be bioadhesive, it must interact with mucus. Mucus is a highly hydrated, viscous anionic hydrogel layer protecting the mucosa. Mucin is composed of flexible glycoprotein chains.

Regarding the mucoadhesive mechanism, the bioadhesive material must come into close contact with the tissue for bioadhesion. Polyacrylic acid polymers, such as Noveon® AA-1 USP polycarbophil, CARBOPOL® polymers and PEMULEN™ polymeric emulsifiers, make excellent bioadhesives. Due to their chemical nature, these high-molecular-weight polymers readily swell in water, providing a large adhesive surface for maximum contact with mucin, the glycoprotein predominant in the mucous layer. Generally, polyacrylic acid polymers interact with the mucin, resulting in adhesion of the polymer to the mucin, although the exact mechanism is not yet fully understood.

The inventive formulations for oral transmucosal drug delivery can include a binder or mixture of two or more binders which facilitate binding of the excipients into a single dosage form. Exemplary binders are selected from the group consisting of cellulosic derivatives (such as methylcellulose, carboxymethyl cellulose, hydroxyethyl cellulose, hydroxyethylmethyl cellulose, etc.), POLYOX™ polyethylene oxide polymers of any molecular weight or grade, irradiated or not, starch, polyvinylpyrrolidone (PVP), AVICEL® microcrystalline cellulose powder, and the like.

The mucoadhesive facilitates prolonged duration of contact between the pharmaceutical composition and the mucus membrane lining the inside of the mouth, including the OLP legions. Upon contact of the pharmaceutical composition with the mucus membrane, moisture in the mucus membrane plasticizes the mucoadhesive, which may then consolidate with the mucus membrane by forming weak bonds with the glycoproteins in the mucus and/or mechanically interlocking with the glycoproteins and lipids in the mucus. The mucoadhesive may increase the residence time of contact of the pharmaceutically active agent and the absorption surface and may facilitate absorption of the pharmaceutically active agents by the absorption surface. A buccal dosage form can contain drug(s), adhesives, and additives and can be formulated as a thin film, a matrix, a reservoir system containing a cavity for the drug(s) and additives separate from the adhesive having an impermeable backing to control direction of drug delivery, to reduce patch deformation and disintegration in use, and to prevent drug loss.

The vehicle functions to facilitate transport and application of the pharmaceutically active agent and mucoadhesive to a legion site. The vehicle can be a liquid vehicle (e.g. a liquid vehicle to form an aqueous dispersion, suspension, or gel) or a solid vehicle (e.g. solid excipient to form the composition into a tablet or lozenge).

Solid excipients can be added to the pharmaceutical composition and then ground and formed into tablets. Examples of solid excipients can include, but are not limited to, sugars, including lactose, sucrose, sucralose, mannitol, or sorbitol; cellulose-based materials, such as corn starch, wheat starch, rice starch, potato starch, gum tragacanth, gelatin, methyl cellulose, polyvinylpyrrolidone, hydroxypropylmethyl-cellulose, and/or sodium caboxymethyl cellulose. Additives to promote disintegration of the solid tablet may include, but are not limited to, agar, alginic acid and/or salts thereof.

The pharmaceutical composition can include other excipients and additives to modify one or more characteristics of the pharmaceutical compositions, such as coating ability, viscosity, palatability, etc, for example. Excipients to improve palatability may include, but are not limited to, sugars, such as lactose, sucrose, sucralose, dextrose, mannitol, or sorbitol; natural sweeteners such as honey; cellulose based additives, such as corn starch, wheat starch, rice starch, and other cellulose additives described above.

The disclosed compositions may optionally include an effective amount of a taste masking agent. A taste-masking agent is one or more agents or compounds that mask or cover any unpleasant or potentially unpleasant taste of one or more composition components, e.g., a steroid, when present in an effective amount. In some embodiments, the compositions may comprise two or more taste masking agents, such as a polyol sweetener and a high intensity sweetener. In some embodiments, the compositions include only a single taste masking agent in the absence of any other sweeteners, flavorants or taste masking agents. In some embodiments, the taste masking agent is (tri)sodium citrate, sodium citrate, sodium chloride, sodium bicarbonate, and combinations thereof. In some embodiments, the taste masking agent is a polyol sweetener. A specific example of one category of polyol sweeteners include sugars, such as dextrose, sucrose, maltose, fructose, and/or lactose. Another specific example of another category of polyol sweeteners include sugar alcohols, e.g., xylitol, sorbitol, mannitol, maltitol, isomaltol, isomalt, erythritol, lactitol, maltodextrin, hydrogenated starch hydrolysates, D-xylose, and/or trehalose. In embodiments, the taste masking agent is a high intensity sweetener or a flavor. A high intensity sweeteners may be sucralose, neotame, aspartame, salts of acesulfame such as the potassium salt of acesulfame (acesulfame K), alitame, saccharin and its salts, cyclamic acid and its salts, glycyrrhizin, dihydrochalcones e.g. neohesperidine DC (NHDCO), thaumatin, monellin, stevioside, and/or aspartame-acesulfame salt. Other examples of suitable taste masking agents include salts of gluconate, such as sodium gluconate. In embodiments, the taste-masking agent is at least one flavoring agents, optionally in combination with one or more food acids. Flavors that can be used include, but are not limited to, coconut, coffee, cola, chocolate, vanilla, orange, lemon, grape fruit, menthol, licorice, anise, apricot, caramel, honey, pineapple, strawberry, raspberry, tropical fruits, cherries, cinnamon, peppermint, wintergreen, spearmint, eucalyptus, and mint flavors. In embodiments, the taste-masking agent in the compositions is sucralose.

Optional viscosity excipients can be added to a liquid formulation of the pharmaceutical composition to modify the flow characteristics of the composition. Flow characteristics can be modified for the purpose of incorporation into a specific application mechanism for applying the composition to a treatment site. Examples of viscosity excipients can include, but are not limited to, glycerine, a carbomer homopolymer, a carbomer copolymer, acacia (gum arabic), agar, aluminum magnesium silicate, sodium alginate, sodium stearate, bladderwrack, bentonite, carbomer, carrageenan, ceratonia, chondrus, dextrose, furcellaran, gelatin, Ghatti gum, guar gum, sterculia gum, gum tragacanth, xanthum gum, hectorite, lactose, maltodextrin, mannitol, sucrose, sorbitol, honey, maize starch, wheat starch, rice starch, potato starch, polyethylene glycols, cellulose, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose, carboxymethyl-cellulose (CMC) (including salts thereof), silicon dioxide, polyvinylpyrrolidone (PVP), Splenda® or combinations thereof.

The pharmaceutical compositions can also include one or more binders, fillers, solvents, lubricants, antioxidants, buffering agents, salts, surfactants, vitamins, pigments, flavorants, disintegrating agents, plasticizers, or combinations thereof. Additional examples of binders can include, but are not limited to any of the aforementioned starches, such as maize starch, wheat starch, rice starch, potato starch, or combinations thereof. Examples of fillers can include, but are not limited to, any of the aforementioned sugars and starches, cellulose, calcium salts, diatomaceous earth, titanium dioxide, other fillers, or combinations thereof. Non-limiting examples of buffers can include, but are not limited to, acetate buffers, citrate buffers, phosphate buffers, other suitable buffers, and combinations thereof.

Examples of antioxidants can include, but are not limited to, ascorbyl palmitate, butylated hydroxyanisole (BHA), butylated hydroxytoluene (BHT), diethylenetriaminepentaacetic acid (DTPA), edetates (EDTA), monothioglycerol, sodium ascorbate, sodium formaldehyde sulfoxylate, sodium metabisulfite, sodium bisulfite, triglycolamate, vitamin E or a derivative thereof, propyl gallate, combinations thereof, or the like.

Surfactants added to the pharmaceutical composition can be anionic, cationic, non-ionic, or zwitterionic. Examples of surfactants can include, but are not limited to, sodium alkyl sulfates (e.g. sodium dodecyl sulfate), quaternary ammonium and pyridinium cationic surfactants, polysorbates, sorbitan esters, bile acids, bile acid salts, nonoxynol or polyoxytheylene glycol fatty acid esters, poloxamers, other pharmaceutically approved surfactants, or combinations thereof. Lubricants can include, but are not limited to, talc, magnesium stearate, of the like, for example. Flavorants can include natural or synthetic flavorants. Plasticizers can include, but are not limited to, glycerol, sorbitol, or the like, for example.

The pharmaceutical compositions disclosed herein can be delivered to the treatment site using various methods. In embodiments, an applicator can be used to apply the compositions to the treatment site. Examples include, but are not limited to, a gel spray bottle with a nozzle, an actuator for a powder puff, a gel tube with an associated applicator, an oral dispensing syringe, or a gel can be administered with an applicator paddle of form a unit dose package. For a spray bottle, the dose could be provided per spray or 2-3 sprays; for thin film, mucoadhesive tablet, polymer mesh, and a unit dose pouch, the dose will be fixed per film, tablet, mesh or pouch respectively. For a syringe, the dose could be per mL dispensed from the syringe. For a gel tube, the dose could be delivered from the calibrated applicator that contains the desired dose. For a gel can, a curved applicator could provide the appropriate dose. For a powder canister, the dose could be provided per puff or per 2-3 puffs.

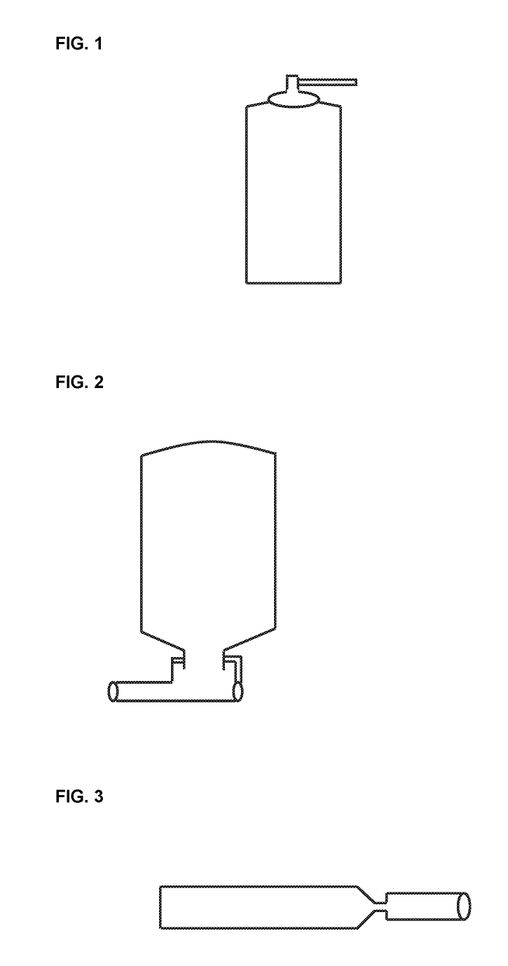

In one embodiment, the pharmaceutical compositions are formed into a gel or aqueous liquid solution capable of being dispensed using a spray bottle having a nozzle capable of targeting the gel or solution to a specific treatment zone within the buccal or sublingual regions of the mouth. A spray bottle for delivering a gel or liquid solution is illustrated in FIG. 1. The spray bottle can include a pump mechanism for pressurizing at least a portion of the aqueous solution or gel containing the pharmaceutical composition. In another aspect, the spray bottle may include a propellant that acts to aerosolize the liquid or gel upon dispensing from the bottle. The nozzle can be configured to create a spray pattern suitable for targeting a treatment zone or can be configured to atomize the liquid or gel into an aerosol. In one embodiment, the nozzle includes an adjustment for adjusting the spray pattern. To use the spray bottle, the nozzle is directed to the area for treatment. The pump mechanism or atomizing nozzle is then activated to dispense the pharmaceutical composition onto the treatment site. The mucoadhesive in the composition contacts the mucus membrane and bonds with the mucus to facilitate contact and absorption of the pharmaceutical agent with and by the absorption surface.

In one aspect, the pharmaceutical composition can be a dry powder composition, and the applicator can be an actuator for delivering a puff of the dry powder to the treatment site. An embodiment of an actuator for delivering a powder puff is illustrated in FIG. 2. The actuator is similar in design and function to an inhaler, except that the powder is dispensed to a treatment site located in the mouth rather than being inhaled into the lungs. The actuator may include an atomizing nozzle and a valve to dispense the dry powder from a canister. The canister may include a propellant to facilitate dispensing and atomization of the pharmaceutical composition. Upon dispensing the dry powder from the actuator, the dry powder composition contacts the mucus membrane in the area of the treatment site and absorbs moisture from the mucus membrane, which wets out or hydrolyzes the dry mucoadhesive. Once hydrolyzed, the mucoadhesive component then consolidates with the mucus membrane to facilitate contact and absorption of the pharmaceutically active agent by the absorption surface.

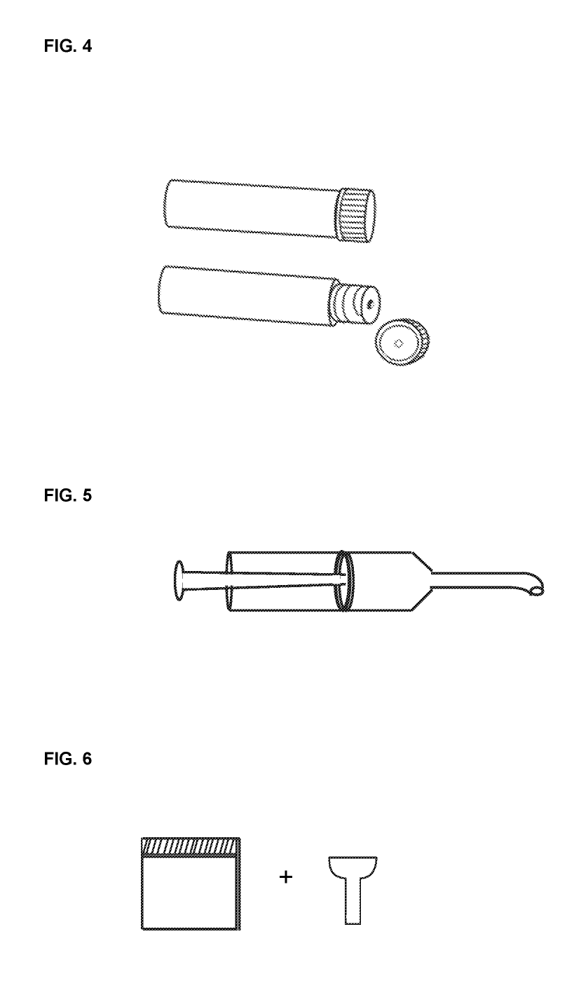

In another aspect, the applicator can include a gel tube with an integral applicator configured to dispense a gel form of the pharmaceutical compositions. An embodiment of a gel tube with integral applicator is illustrated in FIG. 3. The applicator can be integral with the tube or can be configured to be removably engageable with the tube so that the applicator can be discarded after use and another applicator installed on the same tube. The applicator can be configured to meter out a specific dosage of the pharmaceutical composition gel from the tube for each application of the gel to a treatment site. The container may have a child- and/or tamper-proof cap as illustrated in FIG. 4.

In another aspect, the application device can be an oral dispensing syringe that includes a cavity for containing the composition to be administered, a nozzle or tip for directing the composition to the treatment site, and a plunger for dispensing the composition from the oral dispensing syringe. An embodiment of an oral dispensing syringe is illustrated in FIG. 5. The nozzle or tip of the oral dispensing syringe can be curved to facilitate positioning the tip at various buccal and sublingual positions within the mouth. The tip can be flexible to further facilitate positioning the tip within the mouth. The oral dispensing syringe can be made from a polymeric material such as a polyolefin like polyethylene (PE), low density PE (LDPE), high density PE (HDPE), or other thermoplastic material, for example. The cavity of the syringe can be graduated in order to facilitate measuring the desired dosage of the composition to apply to the treatment site.

In another embodiment, the pharmaceutical composition is provided as a gel contained in a container, such as a can, jar, or other vessel for example, and is applied to the treatment site using a paddle applicator, which can be used to smear or spread the gel composition over the treatment site. An embodiment of a container with the paddle applicator is illustrated in FIG. 6. The paddle applicator can be made of any rigid or flexible polymeric material suitable for oral use. Non-limiting examples include polyethylene, HDPE, polyprolylene, other polyolefins, and other polymeric materials.

In addition to applicators to apply a film, paste, gel, liquid, or powder containing the pharmaceutical compositions to the treatment site, the pharmaceutical compositions can be incorporated into a support structure, which can then be applied/adhered to the treatment site. The support structures can be semi-permanent such that the support structure can be applied and then later removed upon exhaustion of the active agents, or the support structures can be dissolvable so that the support structure gradually dissolves as the active agents are absorbed and is ultimately ingested by the user.

Examples of support structures include, but are not limited to a moldable oral bandage, a thin film having the pharmaceutical composition applied to a surface of the film, a biodegradable mucoadhesive drug-loaded polymer mesh, or a mucoadhesive tablet. The pharmaceutical compositions can also be incorporated into one or more of a gum, lozenge, or mouthwash, which can facilitate application of the pharmaceutical agent to the treatment site.

In one aspect, the pharmaceutical compositions can be incorporated into a moldable oral bandage structure that can be applied to the buccal or sublingual regions of the mouth. The pharmaceutical composition can incorporate the corticosteroid into a mixture of water wettable powder that includes a wettable mucoadhesive, such as an alginate (e.g., sodium alginate or potassium alginate), and wettable fibers, such as cellulose fibers. The cellulose fibers may be loose or may be in the form of a sheet that provides an initial structure, planar or other shape, upon which to form the moldable oral bandage. The wettable fibers disposed in a sheet can disassociate upon wetting the composition. The wettable powder can also include a soluble salt reactor, such as calcium sulfate or lead silicate for example, which aid in forming an insoluble alginate salt. The wettable powder can also contain absorbable fillers, such as diatomaceous earth, and/or setting retardants such as sodium or potassium phosphates, oxalates, or carbomates. The wettable powder may also include a starch, such as maize or wheat starch, for example, to aid in handling and shaping the oral bandage upon initial wetting of the wettable powder.

The moldable oral bandage can be applied to a treatment site by first wetting the wettable powder to hydrolyze the mucoadhesive polymer into a tacky gel having a degree of cross-linking of the mucoadhesive polymer. The fibers dispersed in the tacky gel provide reinforcement to the tacky gel so that the gel can be molded or formed into a desired shape of the moldable oral bandage. The molded oral bandage can then be applied to the treatment site, where the mucoadhesive polymer comes into contact with the mucus layer and bonds thereto.

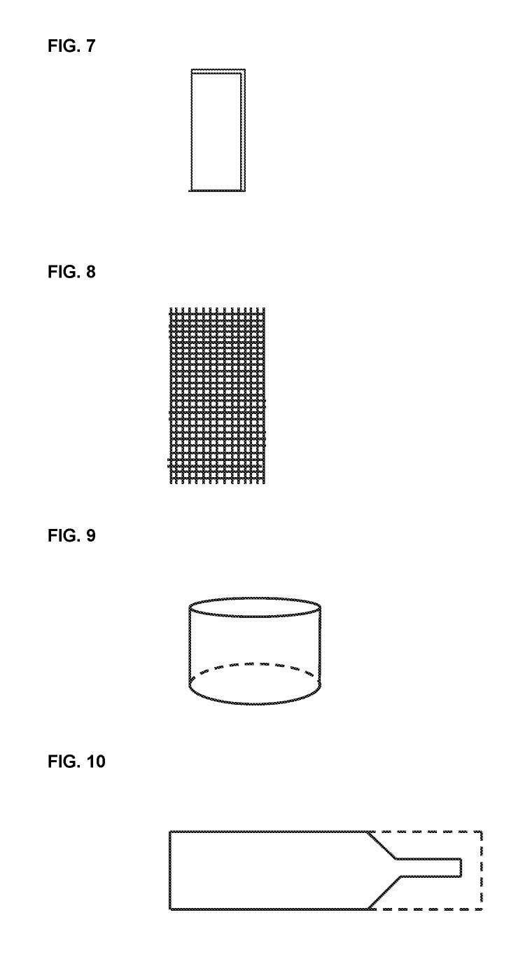

In another aspect, the pharmaceutical compositions can be applied as a thin filmic layer to a surface of a support film to form a drug delivery patch. A patch having the pharmaceutical composition applied to a surface of a support film is illustrated in FIG. 7. The support film can be a thermoplastic film, such as polyolefin or polyester film, for example, or a cellulose based film, such as a thin paper film. The support film can be dissolvable so that the support film can gradually dissolve and be ingested by the user. Examples of dissolvable films can include, but are not limited to cellulose fiber films held together by a weak dissolvable binder, such as starch; polysaccharide polymer films; films formed from starch and pectin; other dissolvable polymer films; or combinations thereof. The pharmaceutical composition, which contains at least the pharmaceutical agent and the mucoadhesive, can be laminated to or coated onto a surface of the support film. The mucoadhesive in the pharmaceutical composition applied to the surface of the film can be wetted before applying to the treatment site, or the pharmaceutical composition can hydrolyze when contacted with moisture in the mucus membrane to which the patch is applied. In one aspect, the pharmaceutical composition can be incorporated into the masterbatch that is extruded or otherwise formed into a biodegradable drug-loaded thin film, which can then be administered to a buccal or sublingual treatment site by adherence of the mucoadhesive to the treatment site or held in the mouth, such as on the tongue, as the thin film gradually degrades and releases the pharmaceutically active agent.

The pharmaceutical compositions can be incorporated into a biodegradable mucoadhesive drug-loaded polymer mesh in addition to the thin film. An embodiment of a biodegradable mucoadhesive drug-loaded polymer mesh is illustrated in FIG. 8. The polymer mesh can be flexible so that it's shape can conform to the contours of the mouth in the buccal and sublingual regions to achieve a more comfortable fit for the user. The polymer mesh can be any of the materials previously described in relation to the thin film. Generally the mesh can be extruded, but the mesh can also be made by extruding filaments, tapes or threads of the support material and weaving the mesh. The pharmaceutical compositions may be incorporated into the polymeric material used to form the mesh or can be coated to an exterior surface of the mesh. In one aspect, an appropriately sized section of the mucoadhesive drug-loaded polymer mesh can be applied directly to the mucus membrane at the treatment site, and the mucoadhesive can be hydrolyzed by moisture from the mucus membrane.

Once hydrolyzed, the mucoadhesive can then bond to the mucus membrane. In another aspect, the mucoadhesive drug-loaded polymer mesh can be pre-wetted to hydrolyze the mucoadhesive prior to applying the polymer mesh to the treatment site.

In one embodiment, the pharmaceutical compositions can include one or more dry solid vehicles to facilitate forming the pharmaceutical compositions into mucoadhesive dissolvable or erodable tablets. A tablet is illustrated in FIG. 9. In one embodiment, pharmaceutical compositions for forming into mucoadhesive tablets may include excipients such as like mannitol and polyethylene glycol (6000). Other examples of bioadhesive or mucoadhesive polymers that may be suitable for tablet embodiments include, but are not limited to, carbopol (e.g. carbopol-934), sodium-carboxymethylcellulose, polyacrylic acid, hydroxymethylcellulose, combinations thereof, and the like. Pharmacologically active agents are added to the mucoadhesive tablet by directly compressing the pharmacologically active agents with the mucoadhesives and any other excipients or additives. In one aspect, the mucoadhesive tablet incorporating the pharmaceutical composition can be applied directly to the mucus membrane at the treatment side, and the mucoadhesive component can be hydrolyzed by moisture from the mucus membrane. Once hydrolyzed, the mucoadhesive polymers can then bond to the mucus membrane. In another aspect, the mucoadhesive at the surface of the tablet can be wetted to hydrolyze a portion of the mucoadhesive prior to applying the tablet to the treatment site.

In one embodiment, the pharmaceutical compositions can be incorporated into a lozenge or gum. For lozenges, the pharmaceutical compositions can be formed into granules and added to a separate lozenge forming composition. The lozenge-forming composition can be a sugar-based composition that can include a single sugar (e.g., sucrose) or a mixture of sugars (e.g., a mixture of sucrose and glucose). The lozenge-forming composition can also be sugar alcohol-based, which can include sorbitol, xylitol, maltitol, maltitol syrup, lactitol, mannitol or mixtures thereof which may be in the form of the free sugar alcohols, derivatives thereof or mixtures thereof. In one aspect, the lozenge-forming composition can include an approximately equimolar mixture of alpha-D-gluco-pyranosyl-1,6-D-sorbitol and alpha-D-glucosopyranosyl-1,1-D-mannitol (isomalt) optionally in conjunction with a hydrogenated glucose syrup such as lycasin. The lozenge-forming composition having the pharmaceutical composition incorporated therein can be heated, molded into desired shapes, and then cooled into individual lozenges. The lozenges may be utilized by placing in the mouth. As the lozenge gradually disintegrates, the pharmaceutical composition is released into the mouth. A lozenge is such that a user may also position the lozenge against one or more treatment sites or may alternate the position of the lozenge between one or more treatment sites to dispense the pharmaceutically active agent directly to the treatment site.

In one embodiment aspect, the pharmaceutical compositions can be added to a gum-based material to form a pharmacologically active chewing gum for treating OLP. The gum-based material can be any suitable natural or synthetic gum and can be generally insoluble. The gum-material can include other softeners, texturizers, or other ingredients to modify the characteristics of the gum. The gum material can be configured to slowly release the pharmaceutical composition from the gum matrix. Like the lozenge embodiment, the gum can be utilized by placing in the mouth and gently chewing the gum. As the user chews the gum, the pharmaceutical composition, including the pharmaceutically active agent, releases from the gum matrix and into the mouth. The user can also position the partially chewed gum against one or more treatment sites or can alternate the position of the partially chewed gum between one or more treatment sites to dispense the pharmaceutically active agent directly to the one or more treatment sites.

In one embodiment, the pharmaceutical compositions can be administered as a mouthwash, which can be swished around the mouth and into the buccal and sublingual regions of the mouth to direct the pharmaceutically active agent to the treatment site. The pharmaceutical compositions can be incorporated into a liquid mouthwash formulation, that includes a liquid vehicle, such as water for example, and one or more additives, such as fluoride, antioxidants, colorants, or other additives. In another aspect, the pharmaceutical compositions can be provided as a dry powder that can be mixed with water or another liquid vehicle by the user at the time of use. Providing the pharmaceutical compositions as a dry powder may facilitate maintaining the activity of and preventing degradation of one or more of the pharmaceutically active agents in the composition.

The following references are expressly incorporated by reference herein in their entirety:

Aleinikov et al. (1996). Topical steroid therapy in oral lichen planus: review of a novel delivery method in 24 patients. J Can Dent Assoc 62(4): 324-327.

Bagan et al. (2012). Topical therapies for oral lichen planus management and their efficacy: a narrative review. Curr Pharm Des 18(34): 5470-5480.

Carbone et al. (2009). Topical clobetasol in the treatment of atrophic-erosive oral lichen planus: a randomized controlled trial to compare two preparations with different concentrations. J Oral Pathol Med 38(2): 227-233.

Eisen et al. (2005). Number V Oral lichen planus: clinical features and management. Oral Dis 11(6): 338-349.

Hearnden et al. New developments and opportunities in oral mucosal drug delivery for local and systemic disease. Advanced Drug Delivery Reviews, 64(2012), 16-28. doi:10.1016/j.addr.2011.02.008

Lavanya et al. (2011). Oral lichen planus: An update on pathogenesis and treatment. Journal of Oral and Maxillofacial Pathology : JOMFP, 15(2), 127-132. http://doi.org/10.4103/0973-029X.84474

Lo Muzio et al. (2001). The treatment of oral aphthous ulceration or erosive lichen planus with topical clobetasol propionate in three preparations: a clinical and pilot study on 54 patients. J Oral Pathol Med 30(10): 611-617.

Rao et al. (2013). Overview on Buccal Drug Delivery Systems. J. Pharm. Sci. & Res. 5, 80-88.

Reddy (2011) A review on bioadhesive buccal drug delivery systems: current status of formulation and evaluation methods. DARU J. Pharmaceutical Sciences 19, 385-403.

Scully et al. (2000). Management of oral lichen planus. Am J Clin Dermatol 1(5): 287-306.

Setterfield et al. (2000). The management of oral lichen planus. Clin Exp Dermatol 25(3): 176-182.

Sudhakar et al. (2006). Buccal bioadhesive drug delivery-A promising option for orally less efficient drugs. J. Controlled Release 114, 15-40.

Thongprasom and Dhanuthai (2008). Steroids in the treatment of lichen planus: a review. J Oral Sci 50(4): 377-385.

The embodiments shown and described in the specification are only specific embodiments of inventors who are skilled in the art and are not limiting in any way. Therefore, various changes, modifications, or alterations to those embodiments may be made without departing from the spirit of the invention in the scope of the following claims. The references cited are expressly incorporated by reference herein in their entirety.

1. A method for delivering a topical steroid to a mucosal surface, the method comprising providing a composition, optionally using a device, the composition comprising at least one bioadhesive polymer and at least one steroid formulated to provide long-acting, sustainable, and optional taste-masking properties, and formulated for use in either acute or chronic administration for therapy of a mucosal pathology.

2. The method of claim 1 where the pathology is lichen planus (LP).

3. The method of claim 1 where the pathology is an aphthous ulcer.

4. The method of claim 1 where the pathology is selected from the group consisting of recurrent aphthous stomatitis (canker sores), a vesiculoerosive disease, glossitis, stomatitis, systemic hypersensitivity to medications, localized inflammatory conditions, pruritis, proctitis, vaginitis, and combinations thereof.

5. The method of claim 1 further providing the steroid in a composition adherent to a mucosal surface.

6. The method of claim 5 where the steroid is not removed by saliva or swallowed before optimal therapeutic effects are achieved.

7. The method of claim 1 where the device, if used, is selected from the group consisting of a spray can, a paddle, a syringe, a film, a spatula, and combinations thereof.

8. A composition comprising at least one bioadhesive polymer containing a steroid, the polymer and steroid formulated in a long-acting preparation, the composition adhering to at least one surface where lesions of lichen planus are or may be present.

9. The composition of claim 8 where the surface is selected from the group consisting cheek, tongue, gum, base of mouth, roof of mouth, and combinations thereof.

10. The composition of claim 8 formulated as a lozenge, a cream, an ointment, a gum, a gel, a foam, a rinse, a lotion, a mouthwash, an oral mucosal surface adherent, and combinations thereof.

11. A steroid delivery formulation for delivery to and/or use on a mucosa or dermatological surface, the formulation selected from the group consisting of a spray, a powder, a puff, a gel, a film, a spread, a solution, a lotion, a cream, an ointment, and combinations thereof.

12. A composition formulated for direct application to a lesion in the oral mucosa, the composition containing at least one oral bioadhesive and at least one taste-masked topical steroid.

13. The composition of claim 12 formulated for application to a lesion from oral lichens planus.

14. The composition of claim 12 further comprising a local anesthetic.

15. An oral mucosal adherent composition comprising at least one bioadhesive and at least one steroid formulated for sustained predictable uniform delivery of the steroid to the mucosa upon application.

16. A method for delivering a topical steroid to the skin, the method comprising providing a composition, optionally using a device, the composition comprising at least one bioadhesive polymer and at least one steroid formulated to provide long-acting, and sustainable properties, and formulated for use in either acute or chronic administration for therapy of a dermatological or mucosal pathology.

17. The method of claim 16 where the pathology is lichen planus (LP).

18. The method of claim 16 further providing the steroid in a composition adherent to at least one of a mucosal or dermatological surface.

19. The method of claim 16 where the device, if used, is selected from the group consisting of a spray can, a paddle, a syringe, a film, a spatula, and combinations thereof.

20. A composition comprising 1.25% w/w CARBOPOL®, 48% w/w glycerin, 0.01% w/w BHA, clobetasol up to 0.05% w/w, and PEG 300 in a formulation for delivery to a mucosal or skin surface under conditions sufficient for treating a mucosal or skin lesion.

21. The composition of claim 20 where clobetasol is at 0.025%.

22. The composition of claim 20 further comprising at least one of a flavorant and a taste-masking agent.

23. The composition of claim 20 where CARBOPOL® is CARBOPOL® 974P.

24. The composition of claim 20 where CARBOPOL® is CARBOPOL® 980.

25. The composition of claim 20 where clobetasol is clobetasol proprionate.
